(19) United States
(12) Patent Application Publication
(10) Pub. No.: US 2020/0206778 A1
(43) Pub. Date: Jul. 2, 2020

(54) MULTILAYER STRUCTURE OF ULTRASONIC PROBE, ULTRASONIC PROBE, AND ULTRASONIC APPARATUS

(71) Applicant: General Electric Company, Schenectady, NY (US)

(72) Inventor: Hiroshi ISONO, Tokyo (JP)

(21) Appl. No.: 16/724,559

(22) Filed: Dec. 23, 2019

(30) Foreign Application Priority Data
Dec. 26, 2018 (JP) 2018-242015

(51) Int. Cl. B06B 1/06; G01N 29/24; A61B 8/00

(52) U.S. Cl. CPC B06B 1/067; A61B 8/4444; G01N 29/2437

(57) ABSTRACT

Object: To provide a multilayer structure of an ultrasonic probe, the multilayer structure being capable of achieving more appropriate acoustic characteristics in accordance with a site from which an ultrasonic image is acquired, a target whose ultrasonic image is to be observed, an object of observing the ultrasonic image, and the like.

Solution: A multilayer structure 2 of an ultrasonic probe includes: a piezoelectric layer 4 from which an ultrasonic wave is emitted to a subject; and a back layer 5 disposed on the piezoelectric layer 4 and opposite the subject across the piezoelectric layer 4, the back layer 5 having an acoustic impedance that is different from an acoustic impedance of the piezoelectric layer 4 within a range from −20% to +20%. The back layer 5 is made of a material including a piezoelectric material or brass. A backing layer 6 is disposed on the back layer 5 and opposite the piezoelectric layer 4 across the back layer 5.

### TECHNICAL FIELD

The present invention relates to a multilayer structure of an ultrasonic probe, the ultrasonic probe, and an ultrasonic apparatus including the ultrasonic probe.

### BACKGROUND ART

A known ultrasonic probe includes a piezoelectric layer, an acoustic matching layer, and a backing layer. The acoustic matching layer is disposed on a side, from which an ultrasonic wave is emitted to a subject, of the piezoelectric layer. The acoustic matching layer achieves a match of an acoustic impedance with the subject. The backing layer is disposed on a back side of an ultrasonic transducer and opposite the subject across the ultrasonic transducer. The backing layer absorbs an unnecessary back echo to efficiently transmit an ultrasonic wave toward the subject. According to this structure, the piezoelectric layer made of a piezoelectric material such as piezoelectric ceramic is sandwiched between the materials lower in acoustic impedance than the piezoelectric material, so that both surfaces of the piezoelectric layer serve as open ends to excite half-wavelength resonance.

Meanwhile, another ultrasonic probe has a dematching structure in which a dematching layer higher in acoustic impedance than the piezoelectric layer is disposed on the back side in place of the backing layer, thereby eliminating an ultrasonic wave thermally consumed at the back side to enhance ultrasonic wave transmission efficiency (refer to, for example, Patent Document 1). According to this structure, the dematching layer with a higher acoustic impedance causes the back side of the piezoelectric layer to serve as a fixed end, so that the fixed end excites quarter-wavelength resonance.

### CITATION LIST

[Patent Document]

[Patent Document 1] U.S. Pat. No. 6,685,647

### SUMMARY OF INVENTION

### Technical Problem

The dematching structure has no heat absorption mechanism by the backing layer, so that an ultrasonic wave is emitted toward only the subject subjected to acoustic matching. Thus, as compared with the half-wavelength resonance backing structure including no dematching layer, the transmission efficiency is improved, and the sensitivity and fractional band in ultrasonic pulse transmission are considerably improved. However, in the dematching structure, although the fractional band is improved, a loop gain of a pulse echo exhibits a flat frequency response. Therefore, as compared with the backing structure, convergence of a real-time waveform is degraded. As described above, since there is a tradeoff relationship between the improvement in the fractional band and the convergence of the real-time waveform, appropriate selection of frequency bandwidth and pulse convergence is required in order to enhance distance resolution particularly in a B-mode image or the like.

In the backing structure, since the half-wavelength resonance is excited, the piezoelectric layer has a thickness that is equal to approximately one-half of a wavelength of an ultrasonic wave to be transmitted. On the other hand, in the dematching structure, since the quarter-wavelength resonance is excited, the piezoelectric layer has a thickness that is equal to approximately one-quarter of a wavelength of an ultrasonic wave to be transmitted. Accordingly, on the assumption that ultrasonic waves of the same frequency are transmitted, the thickness of the piezoelectric layer in the dematching structure is thinner than the thickness of the piezoelectric layer in the backing structure. In a case where the piezoelectric layer is made of a piezoelectric monocrystalline material, the maximum voltage (limit voltage) that does not cause depolarization even when being applied to the piezoelectric layer becomes lower as the piezoelectric layer becomes thinner. Therefore, in the case where the piezoelectric layer is made of a piezoelectric monocrystalline material with limited voltage reliability, the limit voltage in the dematching structure becomes equal to one-half of the limit voltage of the piezoelectric layer in the backing structure including no dematching layer. For this reason, a sound pressure of an ultrasonic pulse transmitted from the ultrasonic probe having the dematching structure is lower than a sound pressure of an ultrasonic pulse transmitted from the ultrasonic probe having the structure including no dematching layer.

As described above, both the ultrasonic probe having the dematching structure and the ultrasonic probe having the structure including no dematching layer have merits and demerits as to acoustic characteristics. It is hence desired to achieve more appropriate acoustic characteristics in accordance with a site from which an ultrasonic image is acquired, a target whose ultrasonic image is to be observed, an object of observing the ultrasonic image, and the like.

### Solution to Problem

In order to solve the problem described above, according to an aspect of the invention, a multilayer structure of an ultrasonic probe includes: a piezoelectric layer from which an ultrasonic wave is emitted to a subject; and a back layer disposed on the piezoelectric layer and opposite the subject across the piezoelectric layer, the back layer having an acoustic impedance that is different from an acoustic impedance of the piezoelectric layer within a range from −20% to +20%.

The acoustic impedance different from the acoustic impedance of the piezoelectric layer within the range from −20% to +20% is an acoustic impedance that lies between an acoustic impedance of a material for a known backing layer and an acoustic impedance of a material for a dematching layer in a known dematching structure, and is also an acoustic impedance that is relatively closer to the acoustic impedance of the piezoelectric layer 4. The term “relatively closer” means that the acoustic impedance is closer to an acoustic impedance of a material for a piezoelectric layer than an acoustic impedance of a material for a backing layer and an acoustic impedance of a material for a dematching layer are.

### Advantageous Effect of Invention

According to the aspect of the invention, the back layer has the acoustic impedance different from the acoustic impedance of the piezoelectric layer within the range from −20% to +20%. It is therefore possible to achieve more appropriate acoustic characteristics as to particularly fractional band, pulse convergence, and sound pressure, in accordance with a site from which an ultrasonic image is acquired, a target whose ultrasonic image is to be observed, an object of observing the ultrasonic image, and the like. In addition, a backing layer is disposed on the back layer and opposite the piezoelectric layer across the back layer, and a known dematching layer is not provided. According to this structure, the piezoelectric layer does not excite quarter-wavelength resonance. Accordingly, a thickness of the piezoelectric layer can be made thicker than an approximately quarter wavelength. It is therefore possible to reduce a possibility of depolarization in a case where the piezoelectric layer is made of a piezoelectric monocrystalline material.

### BRIEF DESCRIPTION OF DRAWINGS

FIG. 1 is a block diagram illustrating an example of an ultrasonic diagnosis apparatus according to an embodiment.

FIG. 2 is a diagram illustrating an example of a multilayer structure in an ultrasonic probe according to the embodiment.

FIG. 3 is a diagram illustrating an example of the multilayer structure in the ultrasonic probe according to the embodiment.

FIG. 4 is a diagram illustrating a thickness of a piezoelectric layer and a thickness of a back layer.

FIG. 5 is a diagram illustrating frequency characteristics of a loop gain of an ultrasonic pulse echo.

FIG. 6 is a diagram illustrating convergence of a real-time waveform of an ultrasonic pulse echo.

FIG. 7 is a diagram illustrating frequency characteristics of a sound pressure of an ultrasonic pulse transmitted in a maximum electric field that causes no depolarization of a piezoelectric layer made of a piezoelectric monocrystalline material.

### DESCRIPTION OF EMBODIMENTS

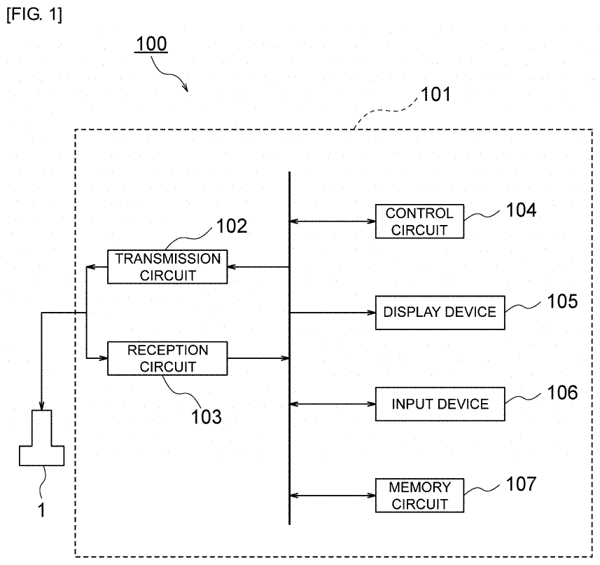

A description will be given of an embodiment of the present invention. An ultrasonic diagnosis apparatus 100 illustrated in FIG. 1 is an example of an ultrasonic apparatus according to an embodiment of the present invention, and displays an ultrasonic image of a subject for the purpose of diagnosis and the like.

The ultrasonic diagnosis apparatus 100 includes an ultrasonic probe 1 and an apparatus main body 101 to which the ultrasonic probe 1 is connected. The apparatus main body 101 includes a transmission circuit 102, a reception circuit 103, a control circuit 104, a display device 105, an input device 106, and a memory circuit 107. The ultrasonic diagnosis apparatus 1 has a configuration as a computer.

The transmission circuit 102 controls transmission of an ultrasonic wave by the ultrasonic probe 1. Specifically, the transmission circuit 102 drives the ultrasonic probe 1 to cause the ultrasonic probe 1 to transmit the various ultrasonic pulses having predetermined parameters, on the basis of control signals from the control circuit 104.

The reception circuit 103 performs signal processing such as phasing addition processing on an ultrasonic echo signal that is transmitted from the ultrasonic probe 1 to the subject, is reflected inside the subject, and is received by the ultrasonic probe 1. The reception circuit 103 performs signal processing on the basis of a control signal from the control circuit 104.

The transmission circuit 102 and the reception circuit 103 may be configured with hardware. However, the ultrasonic diagnosis apparatus 100 does not necessarily include the transmission circuit 102 and the reception circuit 103 as hardware as described above, and instead, may realize the functions of the transmission circuit 102 and reception circuit 103 by software. That is, the control circuit 104 may read a program stored in the memory circuit 107 to execute the functions of the transmission circuit 102 and reception circuit 103.

The control circuit 104 controls the respective components of the ultrasonic diagnosis apparatus, and performs various kinds of signal processing, image processing, and the like. For example, the control circuit 104 performs processing for generating an ultrasonic image on echo data output from the reception circuit 103. The processing for generating an ultrasonic image is processing of generating B-mode data by performing, for example, B-mode processing such as logarithmic compression processing or envelope detection processing. The control circuit 104 scans and converts raw data such as B-mode data with a scan converter to generate ultrasonic image data, and causes the display device 105 to display an ultrasonic image based on the ultrasonic image data.

The control circuit 104 may include, for example, one or more processors. The control circuit 104 may optionally include a central processing unit (CPU), one or more microprocessors, a graphics processing unit (GPU), or any electronic component capable of processing input data in accordance with a specific logic instruction. The control circuit 104 may read a program stored in the memory circuit 107 and execute an instruction. The memory circuit 107 is a tangible non-transitory computer-readable medium to be described later.

The display device 105 is a liquid crystal display (LCD), an organic electro-luminescence (EL) display, or the like.

The input device 106 is a device that accepts operations such as input of an instruction and input of information by an operator. The input device 106 includes a button, a keyboard, and the like that accept input of an instruction or information from the operator, and also includes a pointing device such as a trackball, and the like. The button includes a hard key and a soft key to be displayed on the display device 105. The input device 106 may also include a touch panel. In this case, the button includes a soft key to be displayed on the touch panel.

The memory circuit 107 may be a tangible non-transitory or transitory computer-readable medium such as a flash memory, a hard disk, a RAM, a ROM, and/or an EEPROM. The memory circuit 107 may be used for storing acquired B-mode data, B-mode image data, and color image data that are not scheduled to be displayed immediately, and characters, graphics, and other kinds of data to be displayed on the display device 105.

The memory circuit 107 may also be used for storing firmware or software corresponding to, for example, a graphical user interface, one or more default image display settings, and/or programmed instructions (for, for example, the control circuit 104).

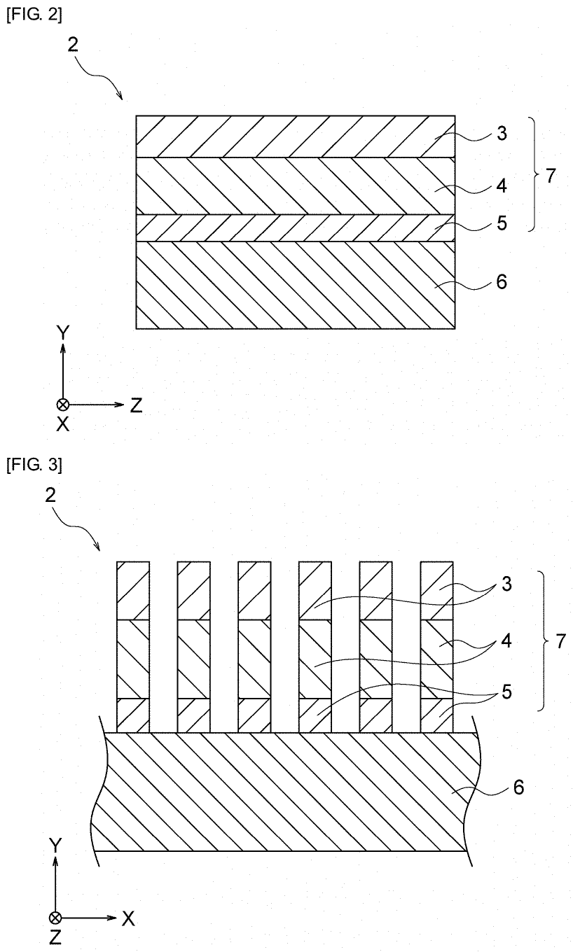

With reference to FIGS. 2 and 3, a description will be given of the ultrasonic probe 1 according to the present example and a multilayer structure 2 of the ultrasonic probe 1. The ultrasonic probe 1 emits an ultrasonic wave to the subject, and receives an ultrasonic echo signal.

The ultrasonic probe 1 includes the multilayer structure 2 including an acoustic matching layer 3, a piezoelectric layer 4, a back layer 5, and a backing layer 6. The multilayer structure 2 is accommodated in a housing (not illustrated) of the ultrasonic probe 1. In the multilayer structure 2, the acoustic matching layer 3, the piezoelectric layer 4, the back layer 5, and the backing layer 6 are layered in a Y-axis direction. The multilayer structure 2 also includes a plurality of layered bodies 7 each including the acoustic matching layer 3, the piezoelectric layer 4, and the back layer 5. The layered bodies 7 are arranged at predetermined spacings in an X-axis direction perpendicular to the Y-axis direction which is a layered direction. The layered bodies 7 arranged in the X-axis direction are disposed on the backing layer 6.

The acoustic matching layer 3 is disposed on one side of the piezoelectric layer 4, and the back layer 5 and the backing layer 6 are disposed on the opposite side of the piezoelectric layer 4 from the side on which the acoustic matching layer 3 is disposed. The side, on which the acoustic matching layer 3 is disposed, of the piezoelectric layer 4 faces toward the subject.

An acoustic lens (not illustrated) is disposed on the acoustic matching layer 3 and opposite the piezoelectric layer 4 across the acoustic matching layer 3. The acoustic matching layer 3 has an acoustic impedance between an acoustic impedance of the acoustic lens and an acoustic impedance of the piezoelectric layer 4. The multilayer structure 2 may include a plurality of layers as the acoustic matching layer 3.

Note that the ultrasonic probe 1 has known configurations (not illustrated) as an ultrasonic probe, in addition to the acoustic lens and the layered bodies 7.

The piezoelectric layer 4 is made of a piezoelectric ceramic material such as lead zirconate titanate (PZT), and an ultrasonic pulse is emitted from the piezoelectric layer 4. The piezoelectric layer 4 may also be made of a piezoelectric monocrystalline material. The piezoelectric layer 4, which is made of, for example, PZT, has an acoustic impedance of 28 to 33 MRayl.

In the multilayer structure 2 according to the present example, unlike a known dematching structure, a resonance structure including a combination of the piezoelectric layer 4 and the back layer 5 cooperatively excites half-wavelength resonance; therefore, adjusting a thickness of the back layer 5 makes a thickness of the piezoelectric layer 4 larger than an approximately quarter wavelength. Here, the thickness of the piezoelectric layer 4 is represented by t1, and the wavelength of the ultrasonic wave to be excited is represented by λ. The following relation is generally established.

λ/4<t1<λ/2

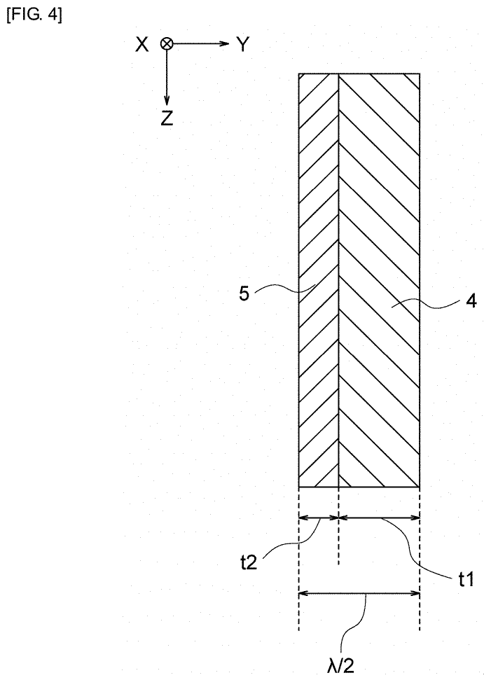

In addition, the thickness of the back layer 5 is represented by t2. A total thickness of the thickness t2 of the back layer and the thickness t1 of the piezoelectric layer 4 is set to generally satisfy λ/2, as illustrated in FIG. 4. That is,

since an equation of t1+t2=λ/2 is established,
an equation of t2=(λ/2)−t1 is established.

The back layer 5 is made of a material having an acoustic impedance different from the acoustic impedance of the piezoelectric layer 4 within a range from −20% to +20%. The acoustic impedance different from the acoustic impedance of the piezoelectric layer 4 within the range from −20% to +20% is an acoustic impedance that lies between an acoustic impedance of a material for a known backing layer and an acoustic impedance of a material for a dematching layer in a known dematching structure, and is also an acoustic impedance that is relatively closer to the acoustic impedance of the piezoelectric layer 4. The term “relatively closer” means that the acoustic impedance of the back layer 5 is closer to the acoustic impedance of the material for the piezoelectric layer 4 than the acoustic impedance of the material for the backing layer and the acoustic impedance of the material for the dematching layer are.

Here, the acoustic impedance of the material for the known backing layer is an acoustic impedance of about 1 MRayl to 10 MRayl, and is an acoustic impedance of about 1/30 to ⅓ relative to the acoustic impedance of the piezoelectric layer 4. Tungsten carbide is also a material for the known dematching layer. Tungsten carbide has an acoustic impedance of about 90 MRayl. The acoustic impedance of tungsten carbide is about three times as large as the acoustic impedance of the piezoelectric layer 4.

Examples of the material for the back layer 5 include a piezoelectric material, brass, and the like.

The backing layer 6 is made of a material having an acoustic impedance of about 1 MRayl to 10 MRayl, as in the material for the known backing layer.

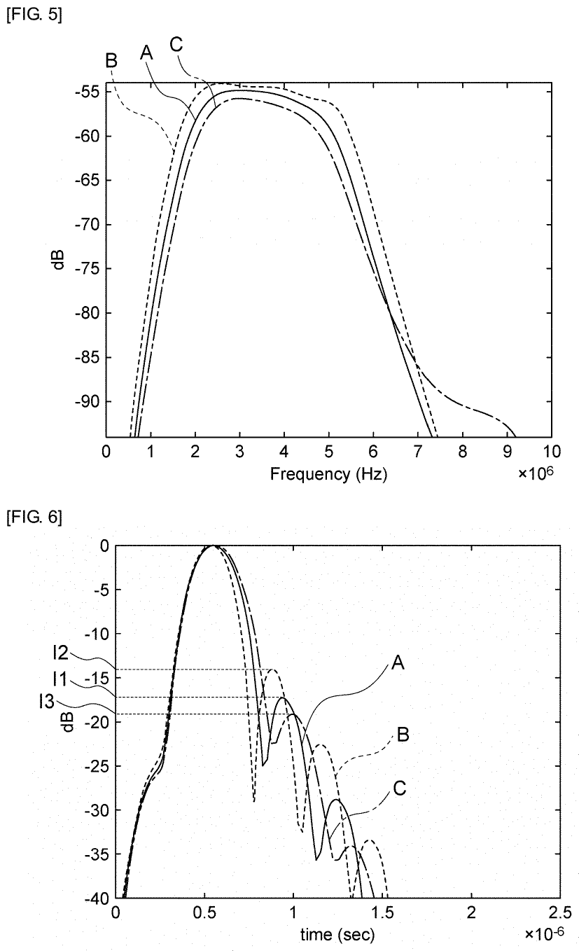

With reference to FIGS. 5 to 7, a description will be given of functions and effects of the multilayer structure 2 according to the present example. FIG. 5 is a diagram illustrating frequency characteristics of a loop gain of an ultrasonic pulse echo. With reference to FIG. 5, a description is given of a fractional band of the multilayer structure 2 according to the present example, the multilayer structure 2 including the back layer 5 disposed on the back side of the piezoelectric layer 4, in a comparison between an ultrasonic probe having a structure including a dematching layer disposed on a back side of a piezoelectric layer (such a structure is referred to as a “dematching structure”) and an ultrasonic probe having a structure including a backing layer disposed on a back side of a piezoelectric layer, but including neither a back layer nor a dematching layer (such a structure is referred to as a “backing structure”).

The dematching structure is a known structure in which a piezoelectric layer excites quarter-wavelength resonance. The backing structure is a known structure in which a piezoelectric layer excites half-wavelength resonance. In FIG. 5, a solid line A indicates the multilayer structure 2 according to the present example, a broken line B indicates the dematching structure, and an alternate long and short dash line C indicates the backing structure.

As illustrated in FIG. 5, the dematching structure has the widest bandwidth, and the multilayer structure 2 according to the present example has a bandwidth between the bandwidth of the dematching structure and the bandwidth of the backing structure. However, the dematching structure is flatter in frequency response characteristic than the other structures, and is poorer in convergence of a real-time waveform than the other structures.

With reference to FIG. 6, a description will be given of convergence of a real-time waveform. FIG. 6 is a diagram illustrating convergence of a real-time waveform of an ultrasonic pulse echo in each of the multilayer structure 2 according to the present example, the dematching structure, and the backing structure. The correspondence relationship between the respective structures and a solid line A, a broken line B, and an alternate long and short dash line C is the same as that illustrated in FIG. 5. The quality of convergence of a real-time waveform is determined on the basis of an echo intensity I of a second peak of multiple peaks in each of the waveform indicated by the solid line A, the waveform indicated by the broken line B, and the waveform indicated by the alternate long and short dash line C in FIG. 6. The higher the echo intensity I is, the poorer the convergence of the real-time waveform is. As illustrated in the figure, an echo intensity I1 of the multilayer structure 2 according to the present example, an echo intensity I2 of the dematching structure, and an echo intensity I3 of the backing structure satisfy a relation of I3<I1<I2. Therefore, as described above, the dematching structure has the poorest convergence of the real-time waveform. On the other hand, the echo intensity I2 of the multilayer structure 2 according to the present example lies between the echo intensity I2 of the dematching structure and the echo intensity I3 of the backing structure.

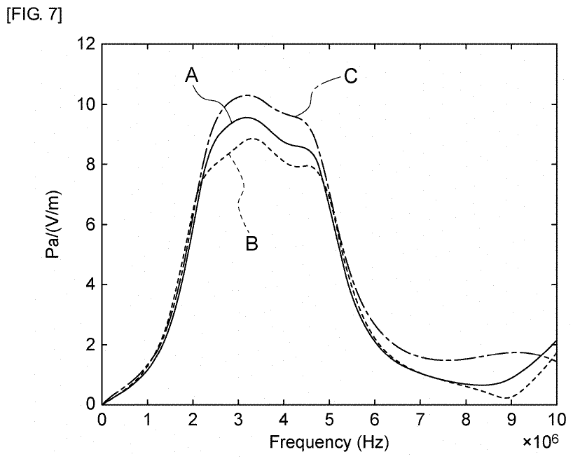

FIG. 7 is a diagram illustrating frequency characteristics of a sound pressure of an ultrasonic pulse transmitted in a maximum electric field that causes no depolarization of a piezoelectric layer made of a piezoelectric monocrystalline material. The vertical axis indicates the sound pressure of the ultrasonic pulse transmitted with the maximum electric field applied to the piezoelectric layer. The correspondence relationship between the multilayer structure 2 according to the present example, the dematching structure, and the backing structure and a solid line A, a broken line B, and an alternate long and short dash line C is the same as those illustrated in FIGS. 5 and 6. As illustrated in the figure, the sound pressure of the dematching structure is minimized as compared with the other structures. On the other hand, the sound pressure of the multilayer structure 2 according to the present example lies between the sound pressure of the dematching structure and the sound pressure of the backing structure.

The acoustic impedance of the back layer 5 lies between the acoustic impedance of the dematching layer and the acoustic impedance of the backing layer, and is relatively closer to the acoustic impedance of the piezoelectric layer 4, that is, the acoustic impedance of the back layer 5 is different from the acoustic impedance of the piezoelectric layer 4 within the range from −20% to +20%. Thus, the acoustic characteristics, that is, the fractional band, pulse convergence, and sound pressure of the multilayer structure 2 according to the present example lie between the characteristics of the dematching structure and the characteristics of the backing structure. In other words, as to the multilayer structure 2 according to the present example, the fractional band is more favorable than that of the known backing structure, and the pulse convergence and sound pressure are more favorable than those of the known dematching structure. According to the present example, it is possible to achieve more appropriate acoustic characteristics in accordance with a site from which an ultrasonic image is acquired, a target whose ultrasonic image is to be observed, an object of observing the ultrasonic image, and the like.

For example, it is required that the pulse convergence and sound pressure are more favorable than the characteristics of the dematching structure depending on a site from which an ultrasonic image is acquired, a target whose ultrasonic image is to be observed, an object of observing the ultrasonic image, and the like. On the other hand, the fractional band is not required as much as the characteristic of the dematching structure in some cases. In such a case, the use of the multilayer structure 2 according to the present example is capable of providing an ultrasonic probe meeting requirements.

In addition, the thickness of the piezoelectric layer 4 can be made thicker than an approximately quarter wavelength. It is therefore possible to reduce a possibility of depolarization in a case where the piezoelectric layer 4 is made of a piezoelectric monocrystalline material, as compared with the dematching structure.

Furthermore, the back layer 5 is made of a piezoelectric material with good processability, which leads to improvement in productivity.

The present invention has been described above on the basis of the foregoing embodiment; however, it goes without saying that various modifications and variations may be made without departing from the scope of the present invention. For example, the layered bodies 7 may be arranged in the X-axis direction and a Z-axis direction perpendicular to the Y-axis direction and perpendicular to each other to constitute a 2D array, a 1.75D array, and a 1.5D array.

### REFERENCE SIGNS LIST

1 Ultrasonic probe
2 Multilayer structure
3 Acoustic matching layer
4 Piezoelectric layer
5 Back layer
6 Backing layer
7 Layered body
100 Ultrasonic diagnosis apparatus

1. A multilayer structure of an ultrasonic probe,
the multilayer structure comprising:
a piezoelectric layer from which an ultrasonic wave is emitted to a subject; and
a back layer disposed on the piezoelectric layer and opposite the subject across the piezoelectric layer,
the back layer having an acoustic impedance different from an acoustic impedance of the piezoelectric layer within a range from −20% to +20%.

2. The multilayer structure of an ultrasonic probe according to claim 1, wherein the back layer is made of a material including a piezoelectric material or brass.

3. The multilayer structure of an ultrasonic probe according to claim 1, wherein the piezoelectric layer is made of a piezoelectric material including a piezoelectric monocrystalline material.

4. The multilayer structure of an ultrasonic probe according to claim 1, further comprising a backing layer disposed on the back layer and opposite the piezoelectric layer across the back layer.

5. The multilayer structure of an ultrasonic probe according to claim 1, further comprising an acoustic matching layer disposed on the piezoelectric layer, the acoustic matching layer facing the subject.

6. The multilayer structure of an ultrasonic probe according to claim 5, comprising:
a plurality of layered bodies each including the acoustic matching layer, the piezoelectric layer, and the back layer,
wherein the layered bodies are disposed on the backing layer to face toward the subject, and are arranged in one direction perpendicular to a layered direction or in two directions perpendicular to the layered direction and perpendicular to each other.

7. An ultrasonic probe comprising the multilayer structure of an ultrasonic probe described in claim 1.

8. An ultrasonic apparatus comprising the ultrasonic probe described in claim 7.
